(19) United States
(12) Patent Application Publication
(10) Pub. No.: US 2016/0114293 A1
(43) Pub. Date: Apr. 28, 2016

(54) Device and Method For Filtering A Suspension

(71) Applicant: Hemacon GmbH, Dusseldorf (DE)

(72) Inventor: OIiver Oechsle, Dusseldorf (DE)

(21) Appl. No.: 14/975,991

(22) Filed: Dec. 21, 2015

Related U.S. Application Data
(62) Division of application No. 13/811,538, filed on Apr. 30, 2013.

(30) Foreign Application Priority Data
Jul. 19, 2010 (DE) 10 2010 031 509.5

(51) Int. Cl. B01D 65/08; A61M 1/34; C02F 1/44; B01D 63/06

(52) U.S. Cl. CPC B01D 65/08; B01D 63/06; A61M 1/34; C02F 1/44; B01D 2321/2008

(57) ABSTRACT

Method for filtering a suspension consisting of a fluid and cell or solid particles, wherein the suspension is guided at least through a curved capillary tube of a filter and passes at least partially through a porous filter wall of the curved capillary tube in order to separate the fluid from the cell or solid particles, wherein the curvature of the capillary tube has a predetermined radius of curvature which is suitable for specifically preventing an accumulation of cell or solid particles of the suspension on an inner curvature edge of the capillary tube.

### REFERENCE TO RELATED APPLICATION

This application is a divisional of U.S. application Ser. No. 13/811,538, filed on Jan. 2, 2013, which is a 35 U.S.C. §371 national stage of and claims priority to PCT/EP2011/062370, filed on Jul. 19, 2011, which claims the benefit of priority to Serial No. DE 10 2010 31 509.5, filed on Jul. 19, 2010 in Germany, the disclosures of which are incorporated herein by reference in their entirety.

### BACKGROUND

The invention relates to a device and a method for filtering a suspension consisting of a fluid and cell or solid particles and in particular to a method for filtering blood and a self-cleaning blood separation filter.

There are various basic methods for separating substance mixtures. These various basic methods include extraction, filtration and distillation.

Extraction is based upon the fact that specific constituents are selectively dissolved out of substance mixtures by means of a solvent and can then be isolated by removal of the solvent. Distillation is a thermal separation method which is based upon the fact that a substance can be removed by evaporation and subsequent condensation of a substance mixture.

In the case of filtration, substance mixtures consisting of solid and liquid substances are separated into their solid and liquid constituents by means of a porous layer which only allows the liquid to pass through. The driving physical force in the case of filtration is the pressure differential—which is produced by the weight of the liquid column located above the filter—between the inlet and outlet side of the respective filter. This pressure differential can be enhanced by pressing on the inlet side or by application of negative pressure on the outlet side or even by centrifugation. Solids having a larger diameter than the pores of the filter material are retained by surface filtration as in the case of a screen. Conventional filters also include capillary filters which consist of one or a plurality of capillary tubes. The capillary tubes consist of a porous material. The wall of the capillary tube forms a cylindrical porous membrane, through which a fluid can pass, whereas solid particles cannot penetrate through the pores.

A disadvantage of this conventional capillary filter resides in the fact that in addition to the main flow which flows in the axial direction through the respective capillary tube, radial secondary flows are produced which cause solid particles to accumulate on the edge of the capillary tubes. As a consequence, the capillary tubes regularly become blocked and must therefore be rinsed with a rinsing agent. This necessary cleaning procedure or rinsing procedure significantly impairs the efficiency of a filter system which uses such capillary filters. The filter procedure must be interrupted in order to rinse the capillary tubes with a rinsing agent as required or at regular intervals. A further disadvantage resides in the fact that in some circumstances the rinsing agent used can lead to contamination.

Therefore, it is an object of the present invention to provide a method and a device for filtering a suspension having solid particles, which avoids or prevents blocking of the capillary tubes without the need for a rinsing procedure.

The invention provides a method for filtering a suspension consisting of a fluid and cell or solid particles, wherein the suspension is guided through at least one curved capillary tube of a filter and passes at least partially through a porous filter wall of the curved capillary tube in order to separate the fluid from the cell or solid particles, wherein the curvature of the capillary tube has a predetermined radius of curvature which is suitable for specifically preventing an accumulation of cell or solid particles of the suspension on an inner curvature edge of the capillary tube.

The suspension can be a liquid substance mixture which has cell or solid particles. In particular, the suspension can be blood which has blood plasma and blood corpuscles.

In the case of a possible embodiment of the method in accordance with the invention, the suspension flowing through the curved capillary tube has blood plasma as a fluid, wherein the filter wall of the capillary tube is formed such that the blood plasma passes at least partially through the filter wall of the capillary tube in order to separate the blood plasma from blood corpuscles. The porosity of the filter wall of the capillary tube is preferably formed such that the fluid flowing through the capillary tube, i.e., the blood plasma, passes at least partially through the pores present in the filter wall in order to separate the fluid, i.e., the blood plasma, from the solid particles, i.e., from the blood corpuscles.

In the case of a possible embodiment of the method in accordance with the invention, a viscous secondary membrane having a high concentration of blood corpuscles, in particular red blood corpuscles, is formed on the outer curvature edge of the curved capillary tube.

The viscous secondary membrane formed on the outer curvature edge of the curved capillary tube causes a change in the flow profile of the blood flowing through the curved capillary tube.

The flow rate of the blood flowing through in the curved capillary tube is increased by reason of the viscous secondary membrane by reason of the smaller flow cross-section which is available for the blood flowing through, and the maximum of the flow profile of the blood flowing through is relocated towards the curvature edge of the capillary tube.

The increased absolute flow rate at the inner curvature edge of the curved capillary tube prevents the formation of a viscous secondary membrane on the inner curvature edge of the curved capillary tube, thus facilitating the passage of blood plasma at the inner curvature edge of the curved capillary tube through the porous filter wall of the curved capillary tube. By virtue of the fact that at the inner curvature edge of the curved capillary tube blood plasma can exit substantially unhindered from a viscous secondary membrane, the volume of the blood plasma, which is separated or filtered from the supplied blood, increases over time. In addition to the absolute increase in the flow rate of the blood flowing through, a maximum of the flow profile is also relocated towards the inner curvature edge of the capillary tube, which means that as a result the formation of a viscous secondary membrane on the inner curvature edge of the curved capillary tube is additionally hampered or prevented.

Accordingly, in the case of a possible embodiment of the method in accordance with the invention, the changed flow profile and the increased flow rate of the blood passing through specifically prevent the formation of a secondary membrane, which consists of blood corpuscles, on the inner curvature edge of the curved capillary tube, thus facilitating at this location the passage of the blood plasma through the porous filter wall of the curved capillary tube to increase the separated quantity or volume of the blood plasma from the blood corpuscles of the blood flowing through, i.e., more blood plasma is filtered out or separated over time.

In the case of a possible embodiment of the method in accordance with the invention, the blood flowing through the curved capillary tube has an increased hematocrit value HK after separation of the blood plasma at the inner curvature edge of the curved capillary tube.

In the case of a further possible embodiment of the method in accordance with the invention, the suspension flowing through the curved capillary tube is formed by a solution which has solid particles, wherein the filter wall of the capillary tube is formed such that the solution passes at least partially through the filter wall of the curved capillary tube for separation of the solid particles, in particular bacteria, cells, fungi or algae.

In the case of a possible embodiment of the method in accordance with the invention, a concentration of the solid particles in the suspension to be filtered or in the filtered suspension is measured by a measuring device.

In the case of a possible embodiment of the method in accordance with the invention, a concentration of blood corpuscles in the blood to be filtered or in the filtered blood is measured by a measuring device.

In the case of a possible embodiment of the method in accordance with the invention, the radius of curvature of the curved capillary tube is adjusted in dependence upon the measured concentration of the solid particles, in particular blood corpuscles, in the suspension to be filtered and/or in the filtered suspension.

In the case of a possible embodiment, the capillary tube or the small capillary tube consists of an elastic material, in particular of an elastic synthetic plastics material.

The plastics material preferably has an elasticity which is adapted to the radius of curvature of the capillary tube. In the case of a possible embodiment, the plastics material is a polyurethane, polyether sulfone or polysulfone.

In the case of a possible embodiment of the method in accordance with the invention, the radius of curvature of the respective capillary tube or small capillary tube can be variably adjusted.

In the case of a possible embodiment of the method in accordance with the invention, the radius of curvature of the capillary tube is adjusted in a range of 1 cm to 25 cm, in particular in a range of 1 cm to 5 cm.

The invention thus provides a self-cleaning filter for filtering a suspension consisting of a fluid and cell or solid particles, having:

at least one capillary tube, through which the suspension flows,

wherein the capillary tube has a filter wall which is formed such that the fluid of the suspension flowing through the capillary tube passes at least partially through the filter wall in order to separate the fluid from the cell or solid particles,

wherein the capillary tube has a curvature which specifically prevents an accumulation of the cell or solid particles on the inner curvature edge of the capillary tube, thus facilitating the passage of the fluid through the filter wall at the inner curvature edge of the capillary tube.

The separated fluid which passes through the filter wall at the inner curvature edge of the capillary tube can be e.g. blood plasma.

The invention thus provides a blood separation filter for filtering blood which has blood corpuscles and blood plasma, having:

at least one capillary tube, through which the blood to be filtered flows,

wherein the capillary tube has a porous filter wall which is formed such that the blood plasma contained in the blood passes at least partially through the filter wall in order to be separated from the blood corpuscles,

wherein the capillary tube has a curvature which prevents formation of a viscous secondary membrane with a high concentration of blood corpuscles on the inner curvature edge of the curved capillary tube, thus facilitating at this location the passage of the blood plasma through the porous filter wall of the capillary tube.

In the case of a possible embodiment of the self-cleaning filter in accordance with the invention, the curved capillary tube has a porous filter wall, whose porosity is formed such that the fluid or blood plasma flowing through the capillary tube passes at least partially through the pores present in the filter wall in order to separate the fluid from the cell or solid particles, in particular from the blood corpuscles.

In the case of a possible embodiment of the self-cleaning filter in accordance with the invention, the capillary tube consists of an elastic plastics material, whose radius of curvature is adjustable.

In the case of a possible embodiment of the self-cleaning filter in accordance with the invention, the suspension to be filtered, in particular the blood, enters the curved capillary tube at a first pressure at a first end and exits the curved capillary tube at a second pressure at a second end in a filtered state, wherein the second pressure is lower than the first pressure.

In the case of a possible embodiment, the exiting filtered suspension is blood having an increased hematocrit value, i.e., having an increased concentration of red blood corpuscles.

In the case of a possible embodiment of the self-cleaning filter in accordance with the invention, the suspension to be filtered is blood which has blood plasma and blood corpuscles and is located in a storage container which is connected to the first end of the curved capillary tube.

In the case of a possible embodiment of the self-cleaning filter in accordance with the invention, the filtered blood exiting at the second end of the curved capillary tube has an increased hematocrit value and is received in a first receiving container.

In the case of a possible embodiment of the self-cleaning filter in accordance with the invention, the blood plasma passing through the porous filter wall of the curved capillary tube against an ambient pressure is received in a second receiving container.

The receiving container for receiving the blood plasma can be a closed container, in which a counter pressure or ambient pressure is specifically built up, in order to adjust the exiting velocity or the volume—exiting over time—of the blood plasma exiting the capillary tube. In the case of a possible embodiment of the self-cleaning filter in accordance with the invention, the concentration of the cell or solid particles in the suspension to be filtered or in the filtered suspension can be measured by a measuring device.

In the case of a possible embodiment of the self-cleaning filter in accordance with the invention, the concentration of blood corpuscles in the blood to be filtered or in the filtered blood can be measured by a measuring device e.g. with reference to a measured hematocrit value.

In the case of a possible embodiment of the self-cleaning filter in accordance with the invention, it has a plurality of arcuate curvatures which are arranged in parallel with one another or in serial fashion.

In the case of a further possible embodiment of the self-cleaning filter in accordance with the invention, at least one capillary tube or a small capillary tube is arranged in a helical manner.

In the case of a possible embodiment of the self-cleaning filter in accordance with the invention, the self-cleaning filter is a blood separation filter for filtering blood which has blood corpuscles and blood plasma, wherein the blood separation filter has a multiplicity of capillary tubes or small capillary tubes which are arranged in parallel and through which the blood to be filtered flows.

Possible embodiments of the inventive self-cleaning filter of the method in accordance with the invention will be explained hereinafter with reference to the accompanying Figures, in which:

### DESCRIPTION OF THE FIGURES

FIG. 1 shows a possible exemplary embodiment of a self-cleaning filter in accordance with the invention;

FIGS. 2A, 2B show further exemplary embodiments of the self-cleaning filter in accordance with the invention;

FIG. 3 shows a further exemplary embodiment of the self-cleaning filter in accordance with the invention;

FIGS. 4A, 4B show further exemplary embodiments of the self-cleaning filter in accordance with the invention;

FIG. 5 shows a further exemplary embodiment of the self-cleaning filter in accordance with the invention;

FIG. 6 shows a further exemplary embodiment of the self-cleaning filter in accordance with the invention;

FIGS. 7A, 7B show diagrams to explain the mode of operation of the self-cleaning filter in accordance with the invention;

FIG. 8 shows a diagram to illustrate a conducted simulation on a capillary tube of the self-cleaning filter in accordance with the invention;

FIGS. 9A, 9B show diagrams to provide evidence of a viscous secondary membrane present in the filter wall of a capillary tube, and of an increased concentration of blood corpuscles in an example of application of the method in accordance with the invention;

FIG. 10 shows a diagram to illustrate a curved capillary tube which has various measurement cross-sections and can be used in the self-cleaning filter in accordance with the invention;

FIGS. 11A, 11B, 12A, 12B, 13A, 13B show velocity profiles in the longitudinal and transverse directions for a suspension flowing through the capillary tube illustrated in FIG. 10;

FIG. 14 shows a block diagram of an exemplary embodiment of a filter system in accordance with the invention,

FIGS. 15A, 15B show diagrams to illustrate the filter method in accordance with the invention.

### DETAILED DESCRIPTION

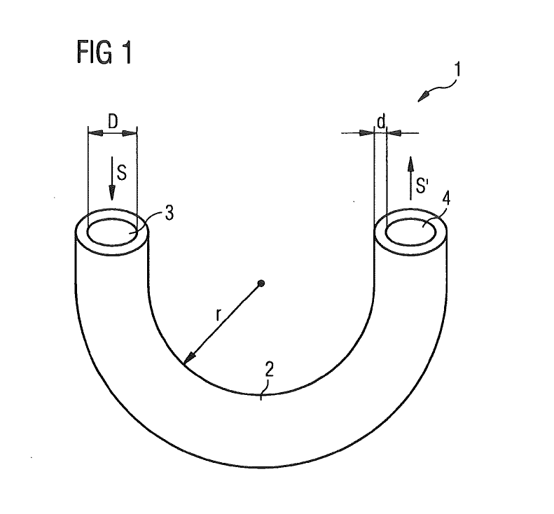

As can be seen in FIG. 1, an inventive, self-cleaning filter 1 for filtering a suspension S has at least one curved capillary tube 2 or small capillary tube. The curvedly arranged capillary tube 2 can have a predetermined radius of curvature r. In the case of a possible embodiment, the radius of curvature r is in a range of 1 cm to 25 cm. The capillary tube 2 has a filter wall with a thickness. The capillary tube 2 filters a suspension S which contains cell or solid particles and a fluid F. In the case of a possible embodiment, the capillary tube 2 filters blood which has blood corpuscles, in particular red blood corpuscles, and blood plasma. The filter wall of the capillary tube 2 is formed such that the suspension S flowing through the capillary tube 2 passes at least partially through the filter wall in order to separate the fluid F from the cell or solid particles. In the case of the example illustrated in FIG. 1, a suspension S which is to be filtered enters an inlet opening 3 and the filtered suspension S′ exits at a second end 4. The capillary tube 2 of the filter 1 has a curvature which prevents an accumulation of solid particles, which blocks the filter 1 or impedes the filter process, on an inner curvature edge of the capillary tube 2. By reason of the curved arrangement of the capillary tube 2, the filter 1 has a self-cleaning function. By reason of the axial flow rate and the radius of curvature r, the cell bodies or solids are acted upon by centripetal forces which are added or subtracted according to the density difference between particle and fluid.

The capillary tube 2 of the filter 1 in accordance with the invention has a porous filter wall. The porosity of the filter wall is formed such that the fluid F of the suspension S flowing through the capillary tube 2 passes at least partially through or out of the pores, which are present in the filter wall, in order to separate the fluid F from the solid particles of the suspension S. The capillary tube 2 illustrated in FIG. 1 can consist of an elastic material. In the case of a possible embodiment, the radius of curvature r of the capillary tube 2 is variably adjustable. In the case of a possible embodiment, the suspension S enters the curved capillary tube 2 at a first pressure P₁ at a first end and exits the capillary tube 2 at a second pressure P₂ at a second end of the curved capillary tube in a filtered state. The second pressure P₂ is lower than the first pressure P₁. In the case of the exemplary embodiment illustrated in FIG. 1, the suspension S to be filtered enters the capillary tube 2 through an inlet opening 3 at a pressure P₁ and exits the capillary tube 2 through the outlet opening 4 at a second low pressure P₂. The curved capillary tube 2 illustrated in FIG. 1 can be arranged horizontally or vertically in a gravitational field, e.g. the gravitational field of the earth.

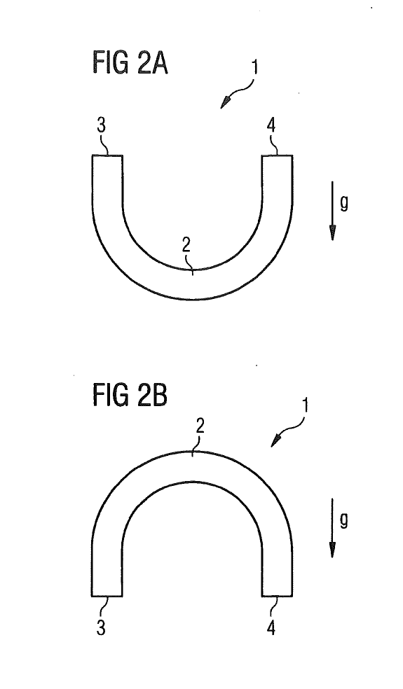

FIGS. 2a, 2b show two different exemplary embodiments for the arrangement of the curved capillary tube 2 vertically with respect to a gravitational field, which exerts a gravitational force g. In the case of the embodiment illustrated in FIG. 2a, the inner curvature edge of the capillary tube 2 points away from the earth's center. In the case of the embodiment illustrated in FIG. 2b, the inner curvature edge of the capillary tube 2 points towards the earth's center. The embodiment illustrated in FIG. 2a offers the advantage that the gravitational force acting upon the cell or solid particles assists the self-cleaning function of the filter 1, since it makes it additionally more difficult for solid particles to accumulate on the curvature edge of the capillary tube 2.

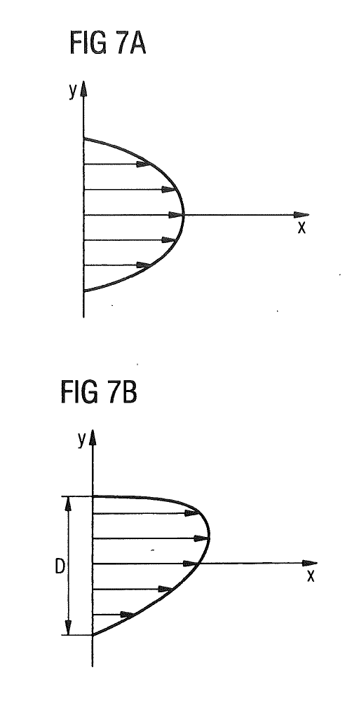

FIGS. 7a, 7b show diagrams to illustrate the self-cleaning function in the case of the filter 1 in accordance with the invention.

FIG. 7a shows a flow profile in the axial direction in the case of a conventional filter having a straight or non-curved capillary tube. The flow direction or axial direction x extends perpendicularly with respect to the cross-sectional direction y of the capillary tube 2. The capillary tube 2 of the filter 1 as illustrated in FIG. 1 has a constant flow diameter D. FIG. 7b shows a flow profile of the filter 1 in accordance with the invention having a curved capillary tube 2. In the case of the diagram illustrated in FIG. 7b, the flow profile has higher flow rates on sides of the inner curvature edge of the capillary tube 2 than on the outer curvature edge of the capillary tube 2. The absolute flow rate depends upon the pressure differential ΔP=P₁−P₂, i.e., upon the pressure differential ΔP between the pressure at the inlet opening 3 and the outlet opening 4 and the local viscosity of the suspension which depends upon the volume ratio of fluid to solid/cells. In a similar manner to a river bed, accumulations of solid particles or sediments can be flushed away by the increased flow or higher flow rate, as can be seen in FIG. 7b, thus hampering or preventing accumulations of solid particles on the inner curvature edge of the capillary tube 2, wherein in particular the formation of a viscous secondary membrane having a high concentration of blood corpuscles with a correspondingly high hematocrit value (HK) on the inner curvature edge of the capillary tube 2 is specifically prevented. This means that the inner curvature edge of the filter wall or the filter surface for separating solids or solid particles is permanently rinsed clean by reason of the curved arrangement of the capillary tube 2 and the filter function of the filter 1 is always maintained without the need for a separate rinsing procedure, e.g. with a rinsing agent.

The narrower the curvature or the smaller the radius of curvature r of the capillary tube 2, the more highly the flow profile illustrated in FIG. 7b is deformed asymmetrically. With a smaller radius of curvature r, the usable surface provided for optimum filtration decreases on the inner curvature arc and the trailing outlet, so that optimum filtration can be optimally selected from increasing radius of curvature r and maximum effective filtration surface.

In the case of a possible embodiment of the filter 1 in accordance with the invention or of the method in accordance with the invention, the pressure P₁ at which the suspension S to be filtered enters the curved capillary tube 2, and the pressure P₂ at which the filtered suspension S′ exits the curved capillary tube 2, is adjustable e.g. by means of pumps. The flow rate at which the suspension S flows through the curved capillary tube 2 can be adjusted in this manner in dependence upon the pressure differential ΔP=P₁−P₂.

Furthermore, in the case of an embodiment of the self-cleaning filter 1 in accordance with the invention, the radius of curvature r of the capillary tube 2 is variably adjustable. In the case of a possible embodiment, the radius of curvature r is variably adjustable in a range of 1 cm to 25 cm. In the case of this embodiment, the capillary tube 2 can consist e.g. of an elastic material. By adjusting the radius of curvature r, centripetal forces can be adjusted corresponding to the flow rates V.

In the case of a possible embodiment, the radius of curvature r and the pressure differential ΔP are adjusted in dependence upon the type of suspension S to be filtered, in particular in dependence upon the viscosity thereof.

In the case of a possible embodiment, the curved capillary tube 2 of the self-cleaning filter 1 as illustrated in FIG. 1 is located in a closed container which serves inter alia to receive the filtered fluid F passing through the filter wall. In the case of a possible embodiment, the ambient pressure P_{U} prevailing in the receiving container is likewise adjustable.

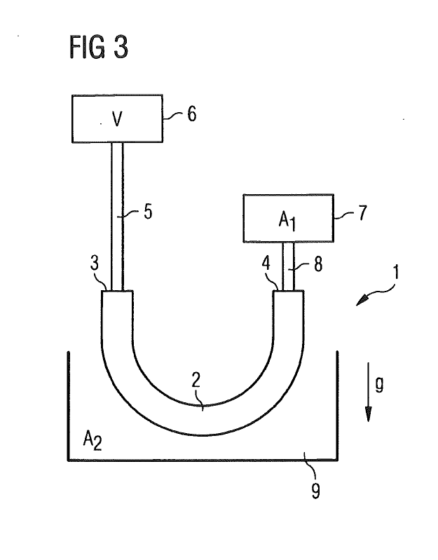

FIG. 3 shows a further exemplary embodiment of the self-cleaning filter 1 in accordance with the invention. In the case of this embodiment, the inlet opening 3 of the capillary tube 2 is connected to a storage container 6 via a tubular line 5. Located in the storage container 6 is the suspension S to be filtered, e.g. donor blood in a donor bag. In the case of the exemplary embodiment illustrated in FIG. 3, the outlet opening 4 at the second end of the curved capillary tube 2 is connected to a first receiving container 7 via a short connection tube 8. The receiving container 7 is e.g. a detachable blood bag which is connected to the outlet opening 4 via a clamp. In the case of the exemplary embodiment illustrated in FIG. 3, the proportion of the suspension S which passes through the filter wall of the curved capillary tube 2 against an ambient pressure P_{U} and has cell or solid particles removed therefrom is received by a second receiving container 9. In the case of the exemplary embodiment illustrated in FIG. 3, the second receiving container 9 is open. In the case of an alternative preferred embodiment, the second receiving container 9 is closed and surrounds the curved capillary tube 2. In the case of the exemplary embodiment illustrated in FIG. 3, the storage container 6 is arranged more highly in a gravitational field than the first receiving container 7, so that as a result a pressure differential ΔP is produced between the inlet opening 3 and the outlet opening 4 of the capillary tube 2.

The filtered suspension S′ exiting at the second end 4 of the curved capillary tube 2 has a higher concentration C′ of cell or solid particles than the suspension S to be filtered entering at the first end 3 of the curved capillary tube 2, or than the substance mixture.

In the case of a possible embodiment, the suspension S flowing into the curved capillary tube 2 is blood and the filter wall of the capillary tube 2 of the filter 1 is formed such that the fluid F, i.e., the blood plasma passes at least partially through the filter wall or filter membrane for separation of blood corpuscles of the blood. In the case of the embodiment illustrated in FIG. 3, this blood plasma is received by the open or closed second receiving container 9. The filtered suspension S′ received in the first receiving container 7 has a higher concentration C′ of blood corpuscles, in particular of red blood corpuscles, and thus has an increased hematocrit value HK.

In the case of a possible embodiment, the filter process performed by the curved capillary tube 2 is performed repeatedly, i.e., the filtered suspension S′ exiting at the outlet opening 4 is guided back e.g. by means of a pump to the inlet opening 3, so that the filter process is repeated. The proportion or concentration C of the cell or solid particles, e.g. the blood corpuscles, present in the filtered suspension S′ increases during each filter process.

The suspension flowing through the curved capillary tube 2 can be a solution, wherein the filter wall is formed such that a fluid of the solution passes at least partially through the filter wall for separation of bacteria, cells, fungi or algae of the suspension S.

The self-cleaning filter 1 in accordance with the invention can be used not only within the field of medicine or in laboratories but can be used for filtering any suspension S which has cell or solid particles. For example, the self-cleaning filter 1 in accordance with the invention is also suitable for cleaning waste water within the field of wastewater treatment plants.

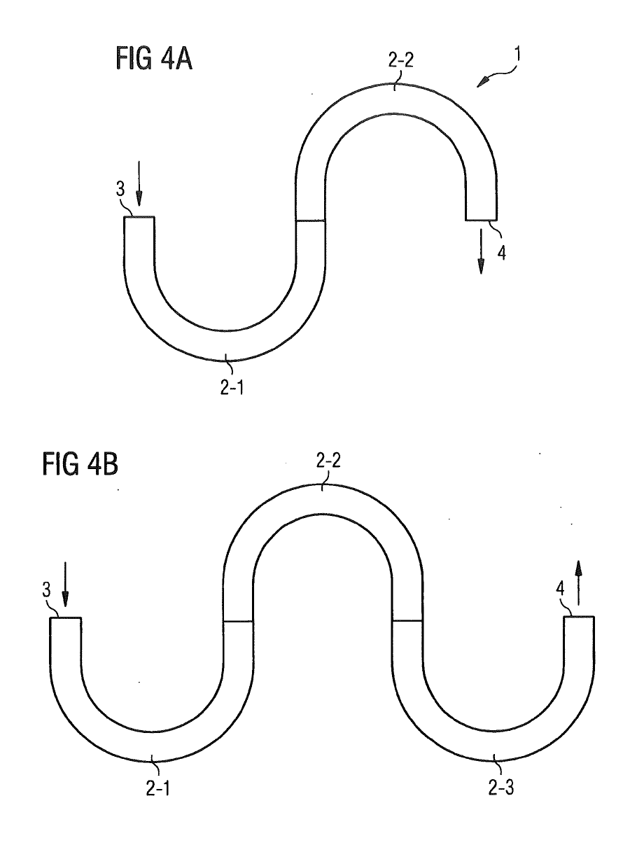

FIGS. 4a, 4b show further exemplary embodiments for the self-cleaning filter 1 in accordance with the invention. In the case of the exemplary embodiments illustrated in FIGS. 4a, 4b, a plurality of curved capillary tubes 2-1, 2-2, 2-3 are connected together in a serial manner. Alternatively, a plurality of capillary tubes 2 can be arranged in parallel with each other and form a bundle of capillary tubes. The capillary tubes 2-i can each be formed by hollow fibers which are produced from plastics material. In the case of a possible embodiment, the plastics material is a hydrophilic material.

In the case of an alternative embodiment, the material, of which the hollow fibers consist, is a hydrophobic material.

The plastics material can be produced by polymerization, polycondensation or polyaddition. Polymerization is the linking of monomers to a double bond to form a macromolecule. In the case of polycondensation, the linking of monomers is effected with separation of a low-molecular substance. Polyaddition is understood to be the linking of molecules without separation of a low-molecular substance. In general, the reaction is effected with migration of a hydrogen atom, wherein chain-like or spatially crosslinked products are obtained. In the case of a possible embodiment, the plastics material of the capillary tube 2 formed by polyaddition is a polyurethane, a polyether sulfone or polysulfone having a high degree of elasticity suitable for the respective radius of curvature r. The smaller the radius of curvature r of the capillary tube 2 is chosen or selected, the greater the elasticity of the plastics material used for the capillary tube 2.

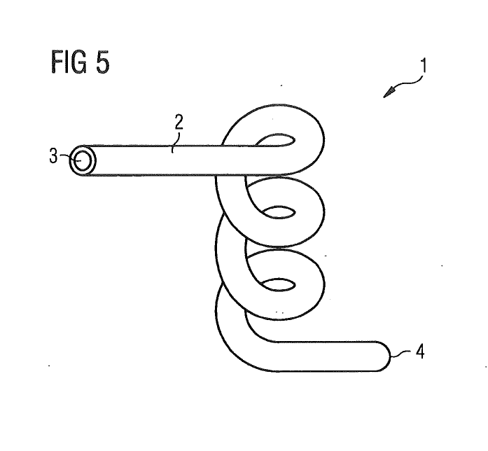

FIG. 5 shows a further exemplary embodiment for a self-cleaning filter 1 in accordance with the invention. In the case of the embodiment illustrated in FIG. 5, the curved capillary tube 2 is arranged in a helical manner. This embodiment offers the advantage that a high number of curvatures can be implemented within a specified volume.

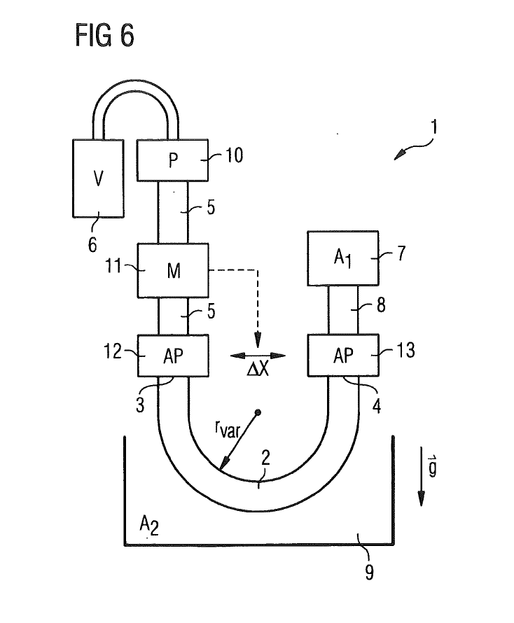

FIG. 6 shows a further exemplary embodiment of a self-cleaning filter 1 in accordance with the invention. In the case of the exemplary embodiment illustrated in FIG. 6, a suspension S which is to be filtered and is located in a storage container 6 is pumped by means of a pump 10 via a measuring device 11 to a curved capillary tube 2. In the case of the embodiment illustrated in FIG. 6, the capillary tube 2 consists of an elastic material. The capillary tube 2 is suspended at its inlet opening 3 and its outlet opening 4 in each case from a suspension point 12, 13 which are spaced apart from each other by a distance Δx in a horizontal direction. Furthermore, a restrictor to regulate the pressure can be provided in the draining line.

In the embodiment illustrated in FIG. 6, the measuring device 11 measures a concentration C of the cell or solid particles, e.g. blood corpuscles, present in the suspension S to be filtered, and automatically adjusts a distance Δx between the suspension points 12, 13 of the curved capillary tube 2 in dependence upon the measured concentration C. By changing the distance Δx of the suspension points 12, 13, the radius of curvature rᵥₐᵣ of the capillary tube 2 changes and is thus variably adjusted. The adjustment of the suspension points 12, 13 can be effected e.g. by actuation of a corresponding motor. In the case of an alternative embodiment, the distance Δx between the suspension points 12, 13 is adjusted manually by a user. The change in the radius of curvature r influences the flow profile of the suspension S flowing through the curved tube 2, as illustrated in FIG. 7b. The flow rate v of the inflowing fluid can be adjusted by means of the pump 10. By adjusting the radius of curvature r, centrifugal forces within the capillary tube 2 can be variably adjusted corresponding to the flow rate v which is present. The filter method in accordance with the invention can be performed by controlling a corresponding control program which runs from a control device, e.g. a microprocessor. In the case of a possible embodiment, this control can measure the concentration C of the cell or solid particles present in the suspension S by means of one or a plurality of measuring devices 11. It is also possible for the increased concentration C′ of cell or solid particles still present in the filtered suspension S′ to be measured. For actuation of pumps, the control program can adjust the pressure differential ΔP between the inlet opening 3 and the outlet opening 4 of the capillary tube 2 in dependence upon the measured particle concentration. In the case of a possible embodiment, not only the pressure differential ΔP but also the radius of curvature r of the capillary tube 2 is adjusted e.g. by changing the distance Δx between the suspension points 12, 13. The adjustment of the pressure differential ΔP and of the radius of curvature r can be effected in dependence upon further parameters, e.g. the type of the respective suspension S, in particular the viscosity thereof. These parameters can be input e.g. via an interface. A display device of the interface can indicate to a user various parameters, in particular the existing pressure differential ΔP, the adjusted radius of curvature r and the measured concentrations c of the entering and exiting suspension S. In the case of the method in accordance with the invention, the filter process does not have to be interrupted in order to clean the filter 1, since the filter 1 is self-cleaning and accumulations of solid particles on the inner curvature edge of the capillary tube 2 are prevented. As a result, the efficiency of a filter system which can use a multiplicity of such capillary tubes 2 can be increased significantly. Furthermore, no rinsing agent is required for a cleaning procedure of the capillary tube 2. The curved capillary tube 2 in accordance with the invention has a filter characteristic, which is constant and does not decline over the course of time, and thus operates in a particularly reliable manner.

The target product supplied by the filter 1 in accordance with the invention can exist both in the filtered fluid F, e.g. blood plasma, which passes out of the capillary tube 2, and also in the filtered suspension S with an increased solid particle concentration C′, e.g. blood with an increased hematocrit value HK, also erythrocyte concentrate. The suspension S can be in particular blood, i.e., blood plasma and blood corpuscles, or other bodily fluids.

Alternatively, the suspension S can also have water or wastewater which contains solid particles. A further example of use is e.g. wine which has solid particles in the form of yeast cells or other particles.

In the case of the self-cleaning filter 1 in accordance with the invention, it is possible to perform an efficient cleaning procedure even with a low pressure differential ΔP between the inlet opening 3 and the outlet opening 4. Particularly within the field of medicine, cells or the like can be destroyed by reason of an excessively high pressure differential ΔP. In the case of the method in accordance with the invention, the low pressure differential ΔP allows cells or blood corpuscles to be obtained or to be preserved undamaged.

By reason of the self-cleaning function of the filter 1 in accordance with the invention, the filter 1 does not need to be blown through, e.g. using a rinsing agent or a gas, under high pressure, so that the filter 1 in accordance with the invention remains sterile or is not contaminated in particular within the field of medicine or in laboratories.

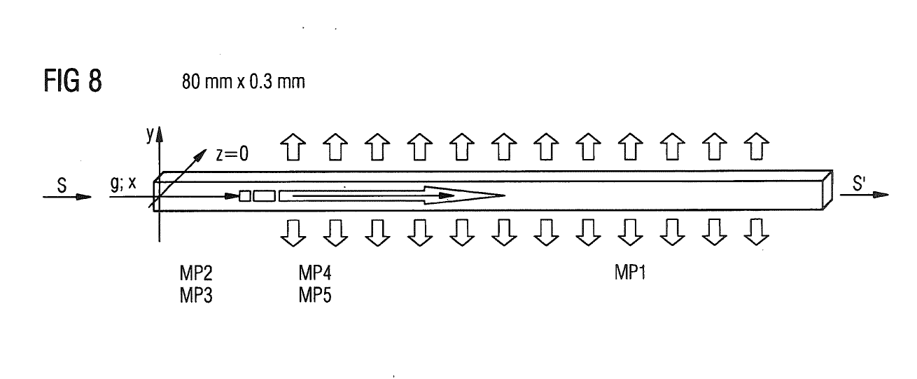

As can be seen in FIG. 8, a filter process can be replicated pursuant to the method in accordance with the invention and the self-cleaning filter 1 in accordance with the invention using a model for numerical simulation. The modeling conducted is based upon a model for multiple-phase flow, in particular blood cells, the concentration of which (volume proportion of cells to plasma) is represented as HKT, and plasma. The transport characteristics or the viscosity of the suspension S as a function of the shear rate and of the HKT can be taken into account. The average velocity through the membrane or the capillary tube 2 amounts in different measurements to 2 μm/s (17.5 TMF) or U1=0.4 μm/s (35 TMF) or U2=0.8 μm/s (70 TMF) (TMF: trans-membrane flow in water in ml/min cm² bar.

From this, it is possible to derive an average flow rate in the axial direction of the suspension S through the capillary tube 2.

In the case of the calculations or simulations, different pressure values and different measuring points MP can be applied, in order to achieve different velocities or flow rates of the suspension, in particular the HKT, e.g. a flow rate of about 2 mm/s. The gravitational force present in the X-direction is preferably taken into account.

In the case of the example illustrated in FIG. 8, 5 measuring or monitor points MP, namely the points MP1, MP2, MP3, MP4, MP5, are illustrated. These monitor points MP are observed in the calculation or simulation. In the illustrated exemplary embodiment, the volume proportion of the suspension or HKT in two regions along the capillary tube 2 at a distance or 5 mm or 20 mm from the inlet or the inlet opening 3 is measured at two measuring points MP which are at a different distance from the capillary wall or filter wall of the capillary tube 2. The velocity of the suspension S is also measured at a measuring point MP1 60 mm downstream of the inflow 3 in the centre of the capillary tube 2, as illustrated in FIG. 8. The coordinates of the monitor or measuring points MP are provided in the flow direction X, wall direction Y in [m] as follows:

MP1: 0.06, 0.0;

MP2: 0.005, 0.00014;

MP3: 0.005, 0.00013;

MP4: 0.02, 0.00014;

MP5: 0.02, 0.00013.

where the X-axis is in the center of the capillary tube 2 and the Y-axis intersects the X-axis at the beginning of the capillary tube 2.

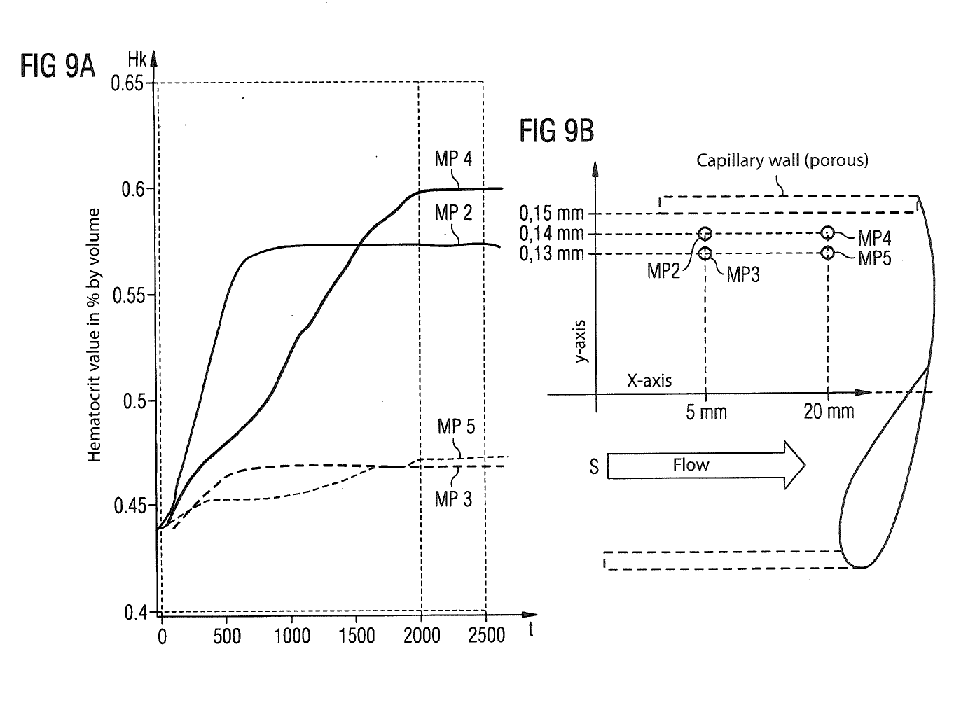

FIG. 9b shows once again the position of the measuring points MP2, MP3, MP4, MP5 at the beginning of the capillary tube 2, through which a suspension S flows, wherein the suspension S is blood. FIG. 9a shows associated measuring results for the various measuring points MP, wherein a hematocrit value HK for the various measuring points MP2, MP3, MP4, MP5 is illustrated in the cumulated time progression. It is clearly apparent from FIG. 9a that the blood plasma passing out of the capillary tube 2 through the porous capillary wall allows the hematocrit value HK, i.e., the concentration of the red blood corpuscles, to increase in the entire capillary tube 2 over time, wherein for the individual measuring points MP different time periods are required until a state of equilibrium is achieved. The measuring points MP3, MP5 which are located 10 μm or 0.01 mm further away on the capillary wall demonstrate a relatively stable progression after a slight increase in the hematocrit value HK, as illustrated in FIG. 9a. The measuring points MP2, MP4 located more closely to the capillary wall of the capillary tube 2 have a considerably higher hematocrit value HK, i.e., the concentration of the blood corpuscles or cell bodies is considerably increased. FIG. 9a clearly shows that in the edge region of the capillary tube 2 in proximity to the capillary wall or filter wall a secondary membrane is building up which has an increased concentration of solids or blood corpuscles. This secondary membrane is viscous and prevents or hampers the penetration of the blood plasma through the porous capillary wall and thus reduces the filtration performance significantly.

The simulation on a curved capillary tube 2 has demonstrated that this viscous secondary membrane is built up downstream of a relatively short straight filtration section on the capillary surface, i.e., on the inner side of the filter wall direction of the capillary tube 2 and thereby reduces the filtration process, i.e., the separated quantity of blood plasma over time. In the curvature arc or in proximity to the apex of the curvature of the capillary tube 2, the viscous secondary membrane lies against the outer curvature edge. There is no viscous secondary membrane located on the inner curvature edge of the curved capillary tube 2 which impairs or hampers the filtering-out of the blood plasma. It is also apparent from the calculation that in the flow direction downstream of the curvature arc the viscous secondary membrane is then dissolved on the outer side of the curvature arc and a secondary membrane then forms on the inner side or on the inner arc. In the case of a curved capillary membrane or a curved capillary tube 2, the filtration process for filtering out a blood plasma thus takes place mainly on the inner curvature edge or in the inner region of the curvature arc and the section of the capillary tube 2 following on directly therefrom.

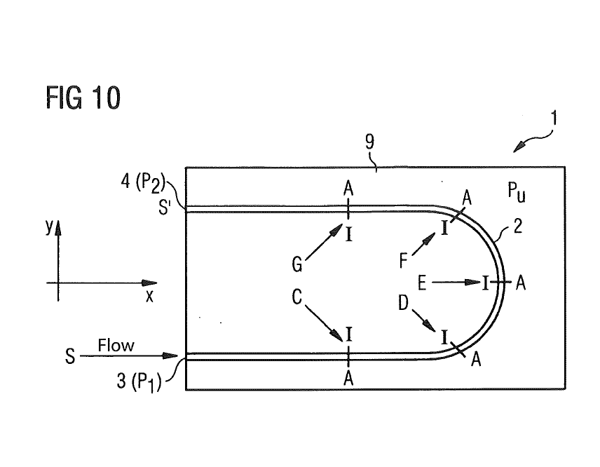

FIG. 10 schematically shows a U-shaped capillary tube 2, into which a suspension S to be filtered enters at an inlet opening 3 and from which a filtered suspension S′ departs at an outlet opening 4. The suspension S can be e.g. blood having a specific concentration C of red blood corpuscles with a corresponding hematocrit value HK0. Filtered blood having an increased concentration C′ of blood corpuscles exits at the outlet opening 4, i.e., the exiting filtered blood S′ has a higher hematocrit value HK′ than the suspension S which entered or the initial blood.

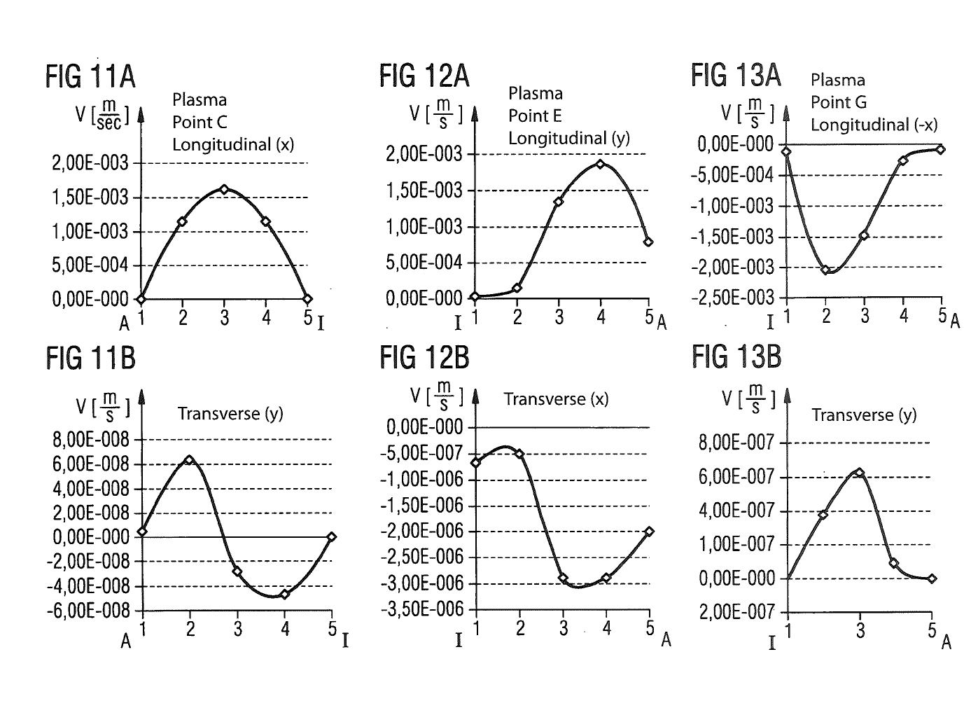

FIGS. 11 to 13 show velocity profiles for the plasma/fluid in the longitudinal and transverse direction for various points or sectional lines C, E and G, through the capillary tube 2, as illustrated in FIG. 10.

As can be seen in FIG. 11a, at point C upstream of the curvature there is a parabolic flow profile in the longitudinal direction X. The associated transverse flow in the Y-direction at point C is illustrated in FIG. 11b. The transverse flow—which is important for the filtration—at the membrane edge of the capillary tube 2 is zero at point C, as is evident in FIG. 11b.

FIG. 12a shows the flow rate at point E, i.e., in proximity to the apex of the curvature in the longitudinal direction Y. As can be seen in FIG. 12a, in the capillary arc, i.e., at the apex E, the parabola of the flow profile is displaced to a considerable extent outwards (A), i.e., in the direction of the outer curvature edge of the capillary tube 2. Furthermore, the absolute flow rate v at the maximum of the flow profile in FIG. 12a is considerably higher than the flow rate v in the case of the parabolic flow profile which is present in the center of the capillary tube 2 at point C, as illustrated in FIG. 11a. At the apex E the transverse flow is also exclusively negative in the X-direction.

At point G of the curved capillary tube 2 illustrated in FIG. 10, the transverse flow at the capillary wall of the capillary tube 2 is negligibly small. However, a flow takes place within the capillary tube 2 for concentration equalization, as can be clearly seen in FIG. 13b. As seen in FIG. 13a, the blood plasma flows in a negative longitudinal direction (−X) towards the outlet opening 4, wherein the flow profile continues to be relocated to the inner curvature edge of the capillary tube 2.

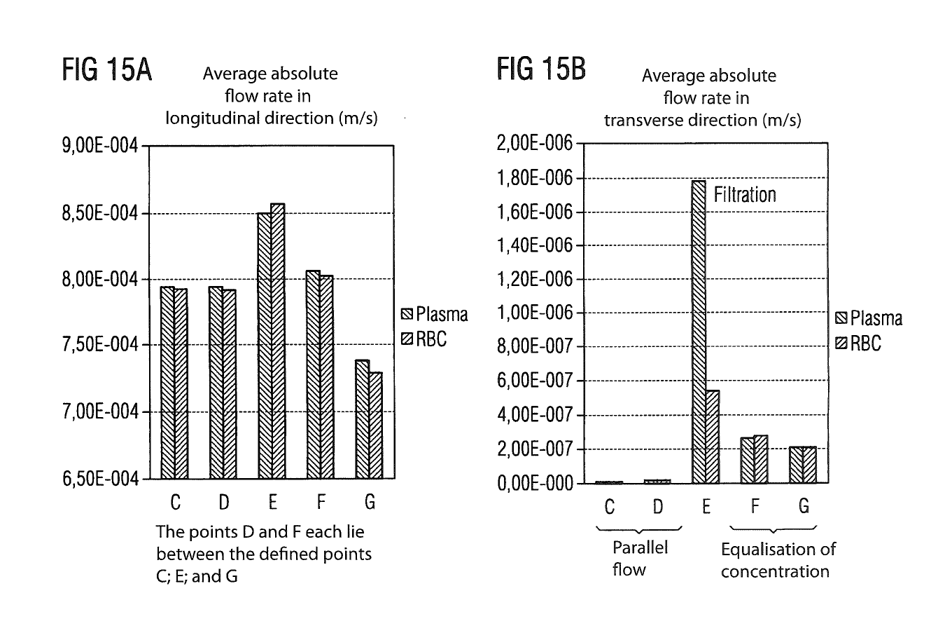

FIG. 15 shows an average absolute flow rate v in the longitudinal direction in [m/s] for the points C, D, E, F, G—illustrated in FIG. 10—for blood plasma as fluid F and RBS. FIG. 10b shows an average absolute flow rate in the longitudinal direction for blood plasma and RBC. It is clearly evident in FIG. 15B that the flow rate in the longitudinal direction is at its maximum at the apex E of the curved capillary tube 2, i.e., that the by far largest quantity of blood plasma is filtered out at this location. Then a concentration equalization takes place, as can be read from the following points in FIG. 15B. Furthermore, the flow rate of the suspension S in the longitudinal direction is likewise at its maximum at the apex E, as can be read from FIG. 15A. Prior to reaching the curvature, the filter performance for separating the fluid, i.e., the blood plasma, is very low by reason of the parallel flow, as can be seen at the points C, D in FIG. 15B.

By changing radius of curvature 2 in the capillary tube 2, it is possible to adjust the flow profile and the absolute flow rate v. Further adjustment parameters are the pressure differential ΔP between the pressure P₁ at the inlet opening 3 and the pressure P₂ at the outlet opening 4 and the adjustable ambient pressure P_{U}, e.g. the pressure inside the receiving container 9 of the separation filter 1. If the ambient pressure P_{U} prevailing around the capillary tube 2 is increased, the quantity of separated blood plasma decreases over time. The entry pressure P₁, at which the suspension S or the blood is injected into the capillary tube 2, and the exit pressure P₂, at which the filtered suspension S′ exits the capillary tube 2, is adjustable. The greater the pressure differential ΔP=P₁−P₂, the higher the pressure gradient present in the capillary tube 2. With the pressure gradient, the flow rate v of the suspension S, e.g. of the blood, inside the capillary tube 2 increases. In the case of a possible embodiment, the self-cleaning filter 1 illustrated in FIG. 10 has a plurality of capillary tubes 2 arranged in parallel, e.g. several hundred capillary tubes or small capillary tubes 2 which are adhered or cast in a sealed receiving container 9 for collecting the blood plasma. In the case of a possible embodiment of the method in accordance with the invention, the parameters, in particular the pressure parameters P₁, P₂ and Pᵤ, are adjusted such that the filtered suspension S′, i.e., the filtered blood, has a specified desired hematocrit value HK desired. In this embodiment, the hematocrit value HK of the filtered blood S′ is thus dependent upon the adjusted pressure parameter values P₁, P₂ and Pᵤ. The hematocrit value HK of the filtered blood S′ can be adjusted in accordance with a medical indication and can be administered to a patient as correspondingly filtered blood S′.

In general, in the case of a possible embodiment of the filter 1 in accordance with the invention, a predetermined desired concentration c desired of solid particles in the filtered suspension S′ can be adjusted or controlled in dependence upon parameters, in particular upon pressure parameters P₁, P₂ and the ambient pressure P_{U}. For example, the concentration of bacteria, cells, fungi or algae in the filtered suspension S′ which exits at the outlet opening 4 can be adjusted in dependence upon the pressure drop ΔP and the ambient pressure P_{U} inside the receiving container 3. The radius of curvature r of the capillary tube 2 can be used as a further adjustment parameter. The injection pressure P₁ can also be adjusted manually, in that an elastic donor bag 6 is compressed accordingly. The ambient pressure P_{U} in a closed elastic receiving container can be increased manually by compression. The concentration C′ of the cell or solid particles in the filtered suspension S′ can be additionally increased by a repeated filter process. The method in accordance with the invention and the filtering device in accordance with the invention is particularly suitable for filtering blood or other bodily fluids. Furthermore, the method in accordance with the invention and the device in accordance with the invention are also suitable for filtering other liquid mixtures which have solid particles.

The method in accordance with the invention and the filter device in accordance with the invention render it possible to adjust the concentration C′ of solid particles at the filter outlet 4 in a specific manner, e.g. by the adjustment of pressure parameters, wherein in addition the curvature of the capillary tube 2 ensures self-cleaning and relatively high filter performance.

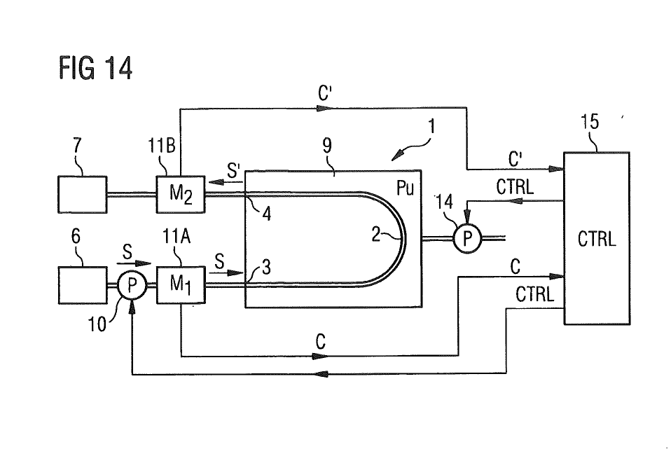

FIG. 14 shows a block diagram of a further exemplary embodiment of a filter system which has at least one self-cleaning filter 1 in accordance with the invention. As can be seen in FIG. 14, the filter system has a control or regulating device 15 which as measurement signals contains a concentration C′ of solid particles of the suspensions entering the filter 1 and as a second measurement signal contains the concentration C′ of solid particles in the filtered, exiting suspension S from the measuring devices 11a, 11b. The two measuring devices 11a, 11b measure e.g. the hematocrit value HK, and thus the blood corpuscle concentration of the entering blood S or of the filtered blood S′. In the illustrated exemplary embodiment, the control or regulating device 15 actuates two pumps 10, 14, in order to adjust or regulate the pressure drop ΔP in the capillary tube 2 and the ambient pressure P_{U} in the receiving container 9. Actuation of the pump 14 serves to increase e.g. the ambient pressure P_{U} in the receiving container 9, in which the curved capillary tube 2 is located. Actuation of the pump 10 serves to increase the entry pressure P₁ and thus the pressure gradient ΔP inside the capillary tube 2. Increasing the ambient pressure P_{U} inside the receiving container 9 serves to reduce the quantity of separated fluid F, e.g. the blood plasma. Separating the blood plasma F in the capillary tube 2 serves to increase the concentration c′ of solid particles in the exiting suspension S′ and makes it higher than the concentration C of solid particles in the entering suspension S. An increase in the ambient pressure P_{U} e.g. by actuation of the pump 14 ensures that less blood plasma F exits into the receiving container 9 through the porous capillary wall of the capillary tube 2 which means that the increase in concentration (ΔC=C′−C) is less than when ambient pressure P_{U} is not increased. If the ambient pressure P_{U} is reduced e.g. by reason of the valve being opened, more fluid or blood plasma is filtered out through the capillary tube 2 and the concentration c′ of the filtered blood increases. As the concentration C′ of the red blood corpuscles within the filtered suspension S′ increases, the hematocrit value HK of the filtered blood which is received by the receiving container 7, e.g. a receiving bag, also increases. In this manner, the control or regulating device 15 can adjust the concentration c′ or the hematocrit value HK′ of the filtered blood S′.

In the case of a possible embodiment, only the concentration c′ of the filtered suspension S′ is measured, which means that the measuring device 11a can be dispensed with. Furthermore, in the case of a possible embodiment, only the ambient pressure P_{U} prevailing in the receiving container 9 is adjusted, which means that the pump 10 can be dispensed with in this embodiment.

The pressure gradient ΔP between the inlet opening 3 and the outlet opening 4 can also be produced e.g. by means of gravitation, in that e.g. a donor bag 6 is suspended at a higher position than the receiving container 7.

The filter system illustrated in FIG. 14 has a self-cleaning separation filter 1. In the case of a further possible embodiment, the filter system has a plurality of self-cleaning separation filters 1 arranged in parallel, in order to obtain a higher volume of filtered suspension S′ within a period of time.

In the case of a further possible embodiment, the filtered fluid F′ is guided back into the entry opening 3, in order to perform a repeated filter process for the purpose of increasing the concentration c′.

The filter method in accordance with the invention is suitable not only for separating blood in plasma and cells but also for separating other liquids, in which solids, cells, particles or the like are to be separated. Examples of use therefore are solutions which contain bacteria, cells, fungi or algae, but also installations for producing drinks, in particular alcoholic drinks such as wine.

The inventive filter process for filtering offers several advantages. The filter 1 which is used is self-cleaning, which means that a separate rinsing procedure is not required. The filtration performance of the filter can be adjusted with the aid of parameters or can be regulated with the aid of measurement data, wherein the parameters include the radius of curvature r, the pressure drop ΔP=P₁−P₂ in the capillary tube 2 and the ambient pressure P_{U} in the receiving container 9 and wherein the measurement data include the concentration values C or e.g. measured hematocrit values HK.

1. A method for filtering a suspension consisting of a fluid and cell or solid particles, wherein:
the suspension is guided at least through a curved capillary tube of a filter and passes at least partially through a porous filter wall of the curved capillary tube in order to separate the fluid from the cell or solid particles, and
the curvature of the capillary tube has a predetermined radius of curvature which is suitable for specifically preventing an accumulation of cell or solid particles of the suspension on an inner curvature edge of the capillary tube.

2. The method as claimed in claim 1, wherein the fluid of the suspension flowing through the curved capillary tube has blood plasma and the filter wall of the capillary tube is formed such that the blood plasma passes at least partially through the filter wall of the capillary tube for separation of blood corpuscles of the suspension.

3. The method as claimed in claim 2, wherein on the outer curvature edge of the curved capillary tube there is formed a viscous secondary membrane which has a high concentration of cell bodies or solids and which changes the flow profile of the suspension flowing through the curved capillary tube such that the flow rate of the suspension flowing through increases and the maximum of the flow profile of the suspension flowing through is displaced towards the inner curvature edge of the capillary tube.

4. The method as claimed in claim 3, wherein the changed flow profile and the increased flow rate of the suspension flowing through prevent the formation of a secondary membrane, consisting of cell bodies or solids, on the inner curvature edge of the curved capillary tube thus facilitating at this location the passage of the fluid through the porous filter wall of the curved capillary tube in order to increase the separation of the fluid from the cell bodies or solids of the suspension flowing through.

5. The method as claimed in claim 4, wherein after separation of the fluid the suspension flowing through the curved capillary tube has an increased cell concentration at the inner curvature edge of the curved capillary tube.

6. The method as claimed in claim 1, wherein:
the suspension flowing through the curved capillary tube is formed by a suspension consisting of cell bodies and solids, of a fluid and bacteria, cells, fungi and algae, and
the filter wall of the capillary tube is formed such that the fluid passes at least partially through the filter wall of the curved capillary tube.

7. The method as claimed in claim 1, wherein a concentration of the cell and solid particles in the suspension to be filtered or in the filtered suspension is measured by means of a measuring device.

8. The method as claimed in claim 7, wherein the radius of curvature of the curved capillary tube is adjusted in dependence upon the measured concentration of the cell and solid particles in the suspension to be filtered and/or in the filtered suspension.

9. The method as claimed in claim 8, wherein the radius of curvature of the capillary tube is adjusted in a range of 1 cm to 5 cm.

10. A filter system for filtering a suspension comprising:
at least one self-cleaning filter including;
at least one capillary tube, through which the suspension flows,
wherein the capillary tube has a filter wall which is formed such that the suspension flowing through the capillary tube passes at least partially through the filter wall in order to separate the fluid from the cell and solid particles, and
wherein the capillary tube has a curvature which specifically prevents an accumulation of solids or cells on the inner curvature edge of the capillary tube, thus facilitating the passage of the fluid through the filter wall at the inner curvature edge of the capillary tube; and
a control device for adjusting filtration parameters of the self-cleaning filter,
wherein the filtration parameters have a first pressure at the inlet of the curved capillary tube, a second pressure at the outlet of the curved capillary tube, an ambient pressure and a radius of curvature of the capillary tube.

11. Use of the filter system as claimed in claim 10 in a filter system, in particular a wastewater treatment plant or a blood filter system, or in a system for producing drinks.
